(19) United States
(12) Patent Application Publication
(10) Pub. No.: US 2026/0232623 A1
(43) Pub. Date: Aug. 13, 2026

(54) USE OF HESPERETIN IN PREPARATION OF DRUG OR FEED ADDITIVE FOR TREATING OR PREVENTING PORCINE ENVELOPED VIRUS INFECTIONS

(71) Applicant: Hubei Horwath Biotechnology Co., Ltd., Xianning City (CN)

(72) Inventors: Yongqing HOU, Xianning City (CN); Tao WU, Xianning City (CN); Ling YANG, Xianning City (CN); Qunbing HU, Xianning City (CN)

(21) Appl. No.: 19/413,421

(22) Filed: Dec. 9, 2025

(30) Foreign Application Priority Data
Dec. 10, 2024 (CN) 202411808796.3

(51) Int. Cl. A61K 31/352; A23K 20/121; A23K 20/195; A61K 9/00; A61K 9/06; A61K 9/16; A61K 47/10; A61K 47/26; A61P 31/14

(52) U.S. Cl. CPC A61K 31/352; A23K 20/121; A23K 20/195; A61K 9/0053; A61K 9/06; A61K 9/1623; A61K 9/1652; A61K 47/10; A61K 47/26; A61P 31/14

(57) ABSTRACT

Use of hesperetin in preparation of a drug or a feed additive for treating or preventing porcine enveloped virus infections is provided. The drug is the hesperetin, or a formulation such as an oral liquid, a granule, a tablet, or a powder prepared by adding the hesperetin as an active ingredient with a solvent or an excipient. The drug is administrated at a dosage of 2 mg to 10 mg per kg of pig body weight; alternatively, the hesperetin is directly added to a feed at 0.01% to 0.03% of a total weight of the feed.

### TECHNICAL FIELD

The present disclosure relates to the technical field of prevention and treatment of porcine enveloped viruses, and specifically relates to use of hesperetin in preparation of a drug or a feed additive for treating or preventing porcine enveloped virus infections.

### BACKGROUND

Porcine enveloped viruses refer to a class of porcine viruses characterized by an envelope structure, which may cause various diseases in pigs. Currently, common porcine enveloped viruses include porcine epidemic diarrhea virus (PEDV), porcine reproductive and respiratory syndrome virus (PRRSV), and African swine fever virus (ASFV), among others. PEDV is the primary cause of porcine epidemic diarrhea (PED). After entering the body via the digestive tract, PEDV binds to receptors, enters cells for replication and proliferation, and counteracts the host's innate immune response, leading to immunosuppression. PEDV primarily induces vomiting and watery diarrhea in neonatal piglets within 10 days of age, resulting in high morbidity and mortality. PRRSV infection leads to porcine reproductive and respiratory syndrome (PRRS). This virus mainly infects porcine alveolar macrophages, causing immune system dysfunction. Key symptoms include low and short-lived neutralizing antibody responses, suppressed cellular immune function, delayed and low expression levels of interferon-γ, and persistent viral carriage in infected pigs, making viral clearance from herds difficult. ASFV is the pathogen responsible for African swine fever (ASF), a highly pathogenic, highly fatal, and highly contagious animal disease. The peracute form has a mortality of up to 100%, such that ASF is classified as a notifiable major animal infectious disease by the World Organization for Animal Health (WOAH).

PEDV, PRRSV, and ASFV have caused significant losses of piglets, resulting in substantial economic damage and severely hindering the development of the swine industry. Currently, disease prevention and control measures against PEDV, PRRSV, and ASFV infections in China primarily rely on vaccines. However, due to the widespread misuse of high doses of antiviral and antibacterial drugs, the mutation rates of viral strains far outpace vaccine development. Additionally, the immunosuppressive characteristics of these viruses lead to suboptimal vaccine efficacy. Furthermore, vaccination only serves a preventive role and cannot protect piglets already infected or showing clinical symptoms. Therefore, relying solely on vaccines generally leads to immunization failures, and large-scale outbreaks of PEDV, PRRSV, and ASFV infections still occur frequently.

In view of the problems associated with existing vaccines in preventing and treating porcine enveloped viruses, developing functional nutrients with anti-porcine enveloped virus properties holds broad application prospects.

### SUMMARY

In view of this, an objective of the present disclosure is to provide use of hesperetin in preparation of a drug or a feed additive for treating or preventing porcine enveloped virus infections. The hesperetin is a natural additive possessing efficacy against porcine epidemic diarrhea virus (PEDV), porcine reproductive and respiratory syndrome virus (PRRSV), and African swine fever virus (ASFV), and is characterized by high safety and convenient application. The hesperetin may be used in the preparation of a drug or a feed additive for treating or preventing porcine enveloped virus infections.

To achieve, or at least partially achieve, the above objective, the present disclosure provides the following technical solutions:

The present disclosure provides use of hesperetin in preparation of a drug or a feed additive for treating or preventing porcine enveloped virus infections.

Compared with the prior art, the technical solutions provided by the present disclosure at least possess the following beneficial effects.
1. In the present disclosure, the hesperetin or a formulation containing the hesperetin may be used as a feed additive for long-term administration to achieve disease prevention. Long-term use does not lead to the generation of drug-resistant strains nor cause other toxic side effects, thereby overcoming drawbacks such as potential immunization failure associated with vaccination and viral mutation caused by the use of antiviral drugs. Feeding pigs with the hesperetin or a formulation containing the hesperetin may enhance the body's antioxidant defense mechanisms, improve the pigs' innate immune function and antiviral activity, and effectively prevent and treat infections caused by PEDV, PRRSV, and ASFV.
2. In the present disclosure, results from in vitro experiments demonstrate that hesperetin has a significant inhibitory effect on the proliferation of viruses such as PEDV, PRRSV, and ASFV.
3. In the present disclosure, results from in vivo experiments indicate that hesperetin enhances innate immune function and antiviral activity, improves intestinal structure and absorption function in pigs, and may effectively prevent and treat infections caused by PEDV, PRRSV, and ASFV, thereby effectively increasing the survival rate and production performance of pigs. When administered orally at 10 mg per kg of pig body weight or added to piglet feed, the hesperetin may achieve a protection rate not less than 90% against infections by PEDV, PRRSV, and ASFV, respectively.

### DETAILED DESCRIPTION OF THE EMBODIMENTS

To make the objectives, technical solutions, and advantages of the present disclosure clearer, the present disclosure is further described in detail below with reference to the drawings and embodiments. It should be understood that the embodiments described herein are merely used to explain the present disclosure, rather than to limit the present disclosure.

Those skilled in the art may understand that, unless specifically stated otherwise, the terms “the”, “this”, and “aforementioned” used in the text of the present disclosure may also include plural forms. It should be further understood that the word “including” used in the specification of the present disclosure refers to the presence of the described features, steps and operations, but does not exclude the presence or addition of one or more other features, integers and steps.

Those skilled in the art may understand that, for experimental steps or conditions not specified in the embodiments, they may be conducted following conventional experimental steps or conditions described in the literature in the relevant field; all raw materials or instrument equipment whose manufacturers are not specified are conventional products that may be obtained commercially.

Those skilled in the art may understand that, unless otherwise stated in the present disclosure, when a numerical range is provided in an embodiment, any of the two endpoints of the range and any value between the two endpoints may be selected. Unless otherwise defined, all technical and scientific terms used in the present disclosure have the same meanings as commonly understood by persons skilled in the relevant art based on their knowledge of the prior art and the descriptions in the present disclosure. Furthermore, the present disclosure may be implemented using any methods, equipment, and materials from the prior art that are similar or equivalent to those described in the embodiments of the present disclosure.

Hesperetin (HES) is a well-known and common flavonoid glycoside used in traditional Chinese medicine (TCM). Its molecular formula is C₁₆H₁₄O₆, and it typically appears as pale yellow needle-like crystals or powder, though it is also described as a beige to light brown crystalline solid. HES is found in relatively high concentrations in medicinal materials such as Fructus Aurantii, Fructus Aurantii Immaturus, and Pericarpium Citri Reticulatae, and predominantly exists in glycoside forms within plants of the Rutaceae family.

In the embodiments of the present disclosure, in vivo and in vitro experiments have revealed that hesperetin and compositions containing the hesperetin may significantly inhibit the proliferation of PEDV, PRRSV, and ASFV, as well as their infection of cells. This hesperetin and these compositions containing the hesperetin (hereinafter referred to as a “hesperetin composition”) may be used in the preparation of drugs or feed additives against PEDV, PRRSV, and ASFV.

Based on this, the embodiments of the present disclosure provide use of hesperetin in preparation of a drug or a feed additive for treating or preventing porcine enveloped virus infections.

In some embodiments, a porcine virus includes one or more of PEDV, PRRSV, and ASFV.

In some embodiments, the drug or the feed additive is the hesperetin, which may be directly used to treat or prevent swine diseases such as PED, PRRS, and ASF.

In some embodiments, the drug or the feed additive is a composition including the hesperetin and an excipient, and the excipient includes one or more of absolute ethanol, water, Tween-80, lactose, defatted rice bran, corn cob powder, soluble starch, wheat bran, silicon dioxide, montmorillonite, vermiculite, maifan stone, a zeolite powder, and white carbon black.

In some embodiments, the composition including the hesperetin is a solution formed by mixing the hesperetin with the absolute ethanol, the Tween-80, and the water as three solvents, and the hesperetin has a concentration of 20 mg/mL to 100 mg/mL.

In some embodiments, the composition including the hesperetin is a solid mixture formed by mixing the hesperetin with the lactose and the soluble starch, and the hesperetin has a mass fraction of 45% to 55% in the solid mixture.

In some embodiments, the composition including the hesperetin is a powdered mixture formed by mixing the hesperetin with the soluble starch or the wheat bran, and the hesperetin has a mass fraction of 45% to 55% in the powdered mixture.

In some embodiments, when the drug or the feed additive is the composition including the hesperetin and the excipient, a dosage form of the composition includes one or more of an oral liquid, a granule, a tablet, and a powder, which may be prepared using conventional pharmaceutical formulation methods.

In some embodiments, a method for administering the drug or the feed additive includes at least one of the following routes:
(1) adding the hesperetin or a composition including the hesperetin to a feed or drinking water for use at a dosage in which a content of the hesperetin is 2 mg to 10 mg per kg of pig body weight;
(2) orally administering the hesperetin or the composition including the hesperetin directly at a dosage in which the content of the hesperetin is 2 mg to 10 mg per kg of pig body weight; and
(3) adding the hesperetin or the composition including the hesperetin to the feed for use at a dosage in which the content of the hesperetin is 0.01% to 0.03% of a total weight of the feed.

The technical solutions and the technical effects achieved by the present disclosure will be described in detail below through more specific examples.

### Example 1

This example provided a method for preparing an oral liquid of hesperetin against porcine enveloped viruses, including the following steps:
(1) 2 g of hesperetin, 20 mL of absolute ethanol, 30 mL of Tween-80 (polyoxyethylene (20) sorbitan monooleate), and a sufficient amount of water for injection were weighed or measured.
(2) The hesperetin was dissolved in the absolute ethanol, then the Tween-80 was added, and finally the water for injection was added slowly to bring a total volume to 100 mL. The mixture was homogenized to obtain a 20 mg/mL hesperetin solution, designated as a hesperetin oral liquid C1.

### Example 2

This example provided a method for preparing an oral liquid of hesperetin against porcine enveloped viruses, including the following steps:
(1) 10 g of hesperetin, 20 mL of absolute ethanol, 30 mL of Tween-80, and a sufficient amount of water for injection were weighed or measured.
(2) The hesperetin was dissolved in the absolute ethanol, then the Tween-80 was added, and finally the water for injection was added slowly to bring a total volume to 100 mL. The mixture was homogenized to obtain a 100 mg/mL hesperetin solution, designated as a hesperetin oral liquid C2.

### Example 3

This example provided a method for preparing hesperetin granules against porcine enveloped viruses, including the following steps:
(1) 50 g of hesperetin, 25 g of lactose, and 25 g of soluble starch were weighed and set aside.
(2) The hesperetin, lactose, and soluble starch were mixed and granulated. The resulting granules were dried to obtain a granular mixture with a hesperetin mass fraction of 50%, designated as hesperetin granules C3.

### Example 4

This example provided a method for preparing a hesperetin powder against porcine enveloped viruses, including the following steps:
(1) 50 g of hesperetin and 50 g of wheat bran were weighed and set aside.
(2) The hesperetin and wheat bran were thoroughly mixed and passed through a 60-mesh sieve to obtain a powdered mixture with a hesperetin mass fraction of 50%, designated as a hesperetin powder C4.

### Example 5

This example provided a method for preparing hesperetin tablets against porcine enveloped viruses, including the following steps:
(1) 50 g of hesperetin, 25 g of lactose, and 25 g of soluble starch were weighed and set aside.
(2) The hesperetin, lactose, and soluble starch were thoroughly mixed, and then compressed into tablets using a tableting machine. The tablets were dried to obtain a tablet mixture with a hesperetin mass fraction of 50%, designated as hesperetin tablets C5.

The formulations and parameters of the hesperetin preparations prepared in the above examples are shown in Table 1:

TABLE 1

| | Prepara- | Prepara- | | Terminalia chebula |
|---|---|---|---|---|
| | tion | tion | | tannin chelate |
| No. | SN | type | Component | concentration |
| Exam- | C1 | Oral liquid | Hesperetin, absolute | 20 mg/mL |
| ple 1 | | | ethanol, Tween-80 | |
| Exam- | C2 | Oral liquid | Hesperetin, absolute | 100 mg/mL |
| ple 2 | | | ethanol, Tween-80 | |
| Exam- | C3 | Granules | Hesperetin, lactose, | 50% (mass fraction) |
| ple 3 | | | soluble starch | |
| Exam- | C4 | Powder | Hesperetin, bran | 50% (mass fraction) |
| ple 4 | | | | |
| Exam- | C5 | Tablet | Hesperetin, lactose, | 50% (mass fraction) |
| ple 5 | | | soluble starch | |

### Inhibitory Effect of Hesperetin on the In Vitro Proliferation of PEDV, PRRSV, and ASFV

The examples of the present disclosure verified the inhibitory effect of the aforementioned hesperetin on the in vitro proliferation of three porcine viruses: PEDV, PRRSV, and ASFV. The verification method was as follows:

### 1. Materials and Methods:

1.1 Viruses: PEDV YN strain, PRRSV, and ASFV were isolated, preserved, and gifted by the National Key Laboratory of Agricultural Microbiology, Huazhong Agricultural University.
1.2 Cells: African green monkey kidney cells (Vero) were used for the proliferation of the PEDV YN strain; monkey embryonic kidney epithelial cells (Marc-145) were used for the proliferation of PRRSV; porcine alveolar macrophages (primary cells) were used for the proliferation of ASFV. Vero and Marc-145 cells were laboratory-preserved cell lines. Porcine alveolar macrophages were isolated from the lungs of healthy piglets.
1.3 Test Agent: Hesperetin (provided by Hubei Horwath Biotechnology Co., Ltd.).
1.4 Reagents: DMEM medium, EDTA, trypsin, fetal bovine serum, and dual antibiotics (penicillin-streptomycin) were all purchased from GIBCO.
1.5 In Vitro Proliferation Inhibition Assay:
(1) PEDV In Vitro Proliferation Inhibition Test: All in vitro tests established three experimental groups and one blank control group. Different doses of hesperetin were added to the culture medium for the experimental groups: a hesperetin low-dose group (added at 20 μg/mL of medium), a hesperetin medium-dose group (added at 40 μg/mL of medium), a hesperetin high-dose group (added at 80 μg/mL of medium), and a blank control group (no hesperetin added to the medium). Vero cells were infected with the PEDV DR13 strain (MOI=0.001), and different doses of hesperetin were added simultaneously with the infection. After 36 h of infection, samples were collected, and the expression levels of the M, N, and S genes of the PEDV DR13 strain were detected by qPCR to verify the inhibitory effect of hesperetin on the in vitro proliferation of the PEDV DR13 strain.
(2) PRRSV In Vitro Proliferation Inhibition Test: All in vitro tests established three experimental groups and one blank control group. Different doses of hesperetin were added to the culture medium for the experimental groups: a hesperetin low-dose group (added at 20 μg/mL of medium), a hesperetin medium-dose group (added at 40 μg/mL of medium), a hesperetin high-dose group (added at 80 μg/mL of medium), and a blank control group (no hesperetin added to the medium). Marc-145 cells were infected with a PRRSV strain (MOI=0.001), and different concentrations of hesperetin were added simultaneously with the infection. After 36 h of infection, samples were collected, and the expression levels of the ORF5 and ORF7 genes of the PRRSV strain were detected by qPCR to verify the inhibitory effect of hesperetin on the in vitro proliferation of the PRRSV strain.
(2) ASFV In Vitro Proliferation Inhibition Test: All in vitro tests established three experimental groups and one blank control group. Different doses of hesperetin were added to the culture medium for the experimental groups: a hesperetin low-dose group (added at 20 μg/mL of medium), a hesperetin medium-dose group (added at 40 μg/mL of medium), a hesperetin high-dose group (added at 80 μg/mL of medium), and a blank control group (no hesperetin added to the medium). Porcine alveolar macrophages were infected with an ASFV strain (MOI=0.001), and different concentrations of hesperetin were added simultaneously with the infection. After 36 h of infection, samples were collected, and the expression levels of the P72 and CD2v genes of the ASFV strain were detected by qPCR to verify the inhibitory effect of hesperetin on the in vitro proliferation of the PRRSV strain.

### 2. Experimental Results:

The intervention effect of hesperetin on the proliferation of the PEDV strain in Vero cells is shown in Table 1 below, where a, b, c indicate significant difference markers.

TABLE 1

| Detected gene | Blank group | 20 μg/mL group | 40 μg/mL group | 80 μg/mL group |
|---|---|---|---|---|
| M | 1.000 ± 0.192^{a} | 0.517 ± 0.069^{b} | 0.479 ± 0.055^{b} | 0.425 ± 0.067^{c} |
| N | 1.000 ± 0.181^{a} | 0.521 ± 0.078^{b} | 0.489 ± 0.079^{b} | 0.419 ± 0.079^{c} |
| S | 1.000 ± 0.197^{a} | 0.531 ± 0.077^{b} | 0.498 ± 0.089^{b} | 0.399 ± 0.058^{c} |

The results in Table 1 show that the addition of hesperetin significantly reduces the expression levels of the N, M, and S genes of PEDV, indicating that hesperetin can significantly reduce the proliferative capacity of PEDV.

The intervention effect of hesperetin on the proliferation of the PRRSV strain in IPAM cells is shown in Table 2 below, where a, b, c indicate significant difference markers.

TABLE 2

| Detected gene | Blank group | 20 μg/mL group | 40 μg/mL group | 80 μg/mL group |
|---|---|---|---|---|
| ORF5 | 1.000 ± 0.101^{a} | 0.498 ± 0.053^{b} | 0.431 ± 0.015^{b} | 0.313 ± 0.052^{c} |
| ORF7 | 1.000 ± 0.099^{a} | 0.502 ± 0.011^{b} | 0.419 ± 0.053^{b} | 0.293 ± 0.011^{c} |

The results in Table 2 show that the addition of hesperetin significantly reduces the expression levels of the ORF5 and ORF7 genes of PRRSV. The addition of hesperetin significantly reduces the proliferative capacity of PRRSV.

The intervention effect of hesperetin on the proliferation of the ASFV strain in porcine alveolar macrophages is shown in Table 3 below, where a, b, c indicate significant difference markers.

TABLE 3

| Detected gene | Blank group | 20 μg/mL group | 40 μg/mL group | 80 μg/mL group |
|---|---|---|---|---|
| P72 | 1.000 ± 0.201a | 0.559 ± 0.076b | 0.463 ± 0.051c | 0.391 ± 0.063d |
| CD2v | 1.000 ± 0.197a | 0.621 ± 0.083b | 0.498 ± 0.073c | 0.413 ± 0.087d |

The results in Table 3 show that the addition of hesperetin significantly reduced the expression levels of the P72 and CD2v genes of ASFV. The addition of hesperetin significantly reduced the proliferative capacity of ASFV.

### Protective Effect of Hesperetin Preparation on PEDV-Infected Piglets

The examples of the present disclosure verified the protective effect of a hesperetin preparation on PEDV-infected piglets. The verification method was as follows:

### 1. Materials and Methods:

1.1 Test Virus: The PEDV YN strain was gifted by the National Key Laboratory of Agricultural Microbiology, Huazhong Agricultural University.
1.2 Test Preparation: Hesperetin oral liquid C1.
1.3 Test Animals: 7-day-old healthy piglets of the same breed and origin, with similar body weights and no maternal antibodies, were selected for the challenge experiment. The test piglets were divided into three experimental groups (low, medium, and high doses) and one blank control group. The hesperetin oral liquid C1 was dissolved in milk, adjusted to a final volume of 3 mL for oral administration. These groups were: the low-dose group (added at a dosage of 2 mg of hesperetin per kg of pig body weight), the medium-dose group (added at a dosage of 5 mg of hesperetin per kg of pig body weight), the high-dose group (added at a dosage of 10 mg of hesperetin per kg of pig body weight), and the blank control group (administered an equivalent dose of milk without the hesperetin preparation). Each group consisted of 10 piglets.
1.4 Challenge Experiment: After 1 week of feeding, piglets in each group were challenged with the PEDV YN strain at a dose of 107 TCID50/mL, with 5.0 mL administered orally per piglet. The control group received an equivalent dose of cell culture medium (DMEM). The piglets were then housed in isolation and observed continuously for 7 d until the end of the experiment.
1.5 Observation Indicators: The diarrhea symptoms of the test piglets were monitored daily. At the end of the experiment, the piglets were slaughtered to observe intestinal lesions. The number of dead piglets was recorded to calculate the diarrhea rate, mortality, and protection rate. The inhibitory effect of the hesperetin preparation on the PEDV was determined by detecting the expression levels of the PEDV structural genes Nor Min the intestinal mucosa of the piglets using real-time quantitative PCR.
1.6 Data Analysis: Experimental data were analyzed using one-way ANOVA and Duncan's multiple range test in SPSS 13.0 statistical software. P<0.05 was considered statistically significant, and P<0.01 was considered highly statistically significant. Results were expressed as mean values.

### 2. Experimental Results:

After adding different doses of hesperetin oral liquid C1 to milk and feeding it to PEDV-infected piglets, the morbidity and mortality of piglets in each group are shown in Table 4, where a, b, c indicate significant difference markers.

TABLE 4

| | Diarrhea | | Protection |
|---|---|---|---|
| Group | rate (%) | Mortality (%) | rate (%) |
| Blank group | 100^{a} | 100^{a} | 0^{b} |
| 2 mg/kg group | 70^{a} | 50^{b} | 50^{c} |
| 5 mg/kg group | 30^{b} | 20^{c} | 80^{a} |
| 10 mg/kg group | 10^{b} | 0^{c} | 100^{a} |

The results in Table 4 show that different doses of hesperetin oral liquid C1 significantly reduces the diarrhea rate and mortality of PEDV-infected piglets. Compared with the blank control group, the diarrhea rate in PEDV-infected piglets decreases to 10% to 70%, and the mortality among diseased piglets decreases to 0% to 50%. The experimental results fully demonstrate that the addition of the hesperetin preparation significantly increases the protection rate of piglets against PEDV infection.

The expression levels of the PEDV structural genes M, N, and S in the intestinal mucosa of the jejunum, ileum, and colon tissues of the piglets were detected. The detection results are shown in Table 5 below, where a, b, c, d indicate significant difference markers.

TABLE 5

| Sampled | Detected | Blank | 2 mg/kg | 5 mg/kg | 10 mg/kg |
|---|---|---|---|---|---|
| tissue | gene | group | group | group | group |
| Jejunum | M | 1.000 ± 0.256^{a} | 0.238 ± 0.049^{b} | 0.156 ± 0.03^{c} | 0.121 ± 0.051^{c} |
| | N | 1.000 ± 0.281^{a} | 0.260 ± 0.053^{b} | 0.146 ± 0.047^{c} | 0.138 ± 0.035^{c} |
| | S | 1.000 ± 0.191^{a} | 0.366 ± 0.074^{b} | 0.161 ± 0.046^{c} | 0.145 ± 0.036^{c} |
| Ileum | M | 1.000 ± 0.238^{a} | 0.599 ± 0.153^{b} | 0.298 ± 0.142^{c} | 0.231 ± 0.137^{d} |
| | N | 1.000 ± 0.266^{a} | 0.444 ± 0.112^{b} | 0.311 ± 0.098^{c} | 0.232 ± 0.099^{d} |
| | S | 1.000 ± 0.281^{a} | 0.630 ± 0.124^{b} | 0.278 ± 0.118^{c} | 0.258 ± 0.132^{d} |
| Colon | M | 1.000 ± 0.260^{a} | 0.273 ± 0.080^{b} | 0.187 ± 0.079^{c} | 0.168 ± 0.079^{d} |
| | N | 1.000 ± 0.210^{a} | 0.484 ± 0.104^{b} | 0.199 ± 0.097^{c} | 0.175 ± 0.095^{d} |
| | S | 1.000 ± 0.219^{a} | 0.367 ± 0.104^{b} | 0.201 ± 0.101^{c} | 0.198 ± 0.091^{d} |

The results in Table 5 show that different doses of hesperetin oral liquid C1 significantly reduce the expression levels of the N, M, and S genes of PEDV, indicating that the addition of the hesperetin preparation significantly inhibites the proliferative capacity of the PEDV virus within the pig intestines.

### Protective Effect of Hesperetin Preparation on PRRSV-Infected Piglets

The examples of the present disclosure verified the protective effect of a hesperetin preparation on PRRSV-infected piglets. The verification method was as follows:

### 1. Materials and Methods:

1.1 Test Virus: The PRRSV strain was gifted by the National Key Laboratory of Agricultural Microbiology, Huazhong Agricultural University.
1.2 Test Preparation: Hesperetin powder C4.
1.3 Test Animals: 25-day-old healthy piglets of the same breed and origin, with similar body weights and no maternal antibodies, were selected for the challenge experiment. The test piglets were divided into three experimental groups (low, medium, and high doses) and one blank control group. The hesperetin powder was dissolved in milk, adjusted to a final volume of 3 mL for oral administration. These groups were: the low-dose group (added at a dosage of 2 mg of hesperetin per kg of pig body weight), the medium-dose group (added at a dosage of 5 mg of hesperetin per kg of pig body weight), the high-dose group (added at a dosage of 10 mg of hesperetin per kg of pig body weight), and the blank control group (administered an equivalent dose of milk without the hesperetin preparation). Each group consisted of 10 piglets.
1.4 Challenge Experiment: After 1 week of feeding, piglets in each group were challenged with the PRRSV strain at a dose of 107 TCID50/mL, with 5 mL administered orally per piglet. The control group received an equivalent dose of physiological saline. The piglets were then housed in isolation and observed continuously for 7 d until the end of the experiment.
1.5 Observation Indicators: The condition of the test piglets was observed daily. At the end of the experiment, the piglets were slaughtered to observe pathological lesions in lung tissues. The number of dead piglets was recorded to calculate the morbidity and mortality. The inhibitory effect of hesperetin on PRRSV was determined by detecting the expression level of the PRRSV ORF7 gene in the brain and lungs of the piglets using real-time quantitative PCR.
1.6 Data Analysis: Experimental data were analyzed using one-way ANOVA and Duncan's multiple range test in SPSS 13.0 statistical software. P<0.05 was considered statistically significant, and P<0.01 was considered highly statistically significant. Results were expressed as mean values.

### 2. Experimental Results:

After adding different doses of hesperetin powder C4 to milk and feeding it to PRRSV-infected piglets, the morbidity and mortality of piglets in each group are shown in Table 6, where a, b, c indicate significant difference markers.

TABLE 6

| Group | Morbidity (%) | Mortality (%) | Protection rate (%) |
|---|---|---|---|
| Blank group | 100^{a} | 80^{b} | 20^{c} |
| 2 mg/kg group | 60^{a} | 40^{b} | 60^{a} |
| 5 mg/kg group | 30^{b} | 10^{c} | 90^{a} |
| 10 mg/kg group | 20^{b} | 10^{c} | 90^{a} |

The results in Table 6 show that oral administration of different doses of hesperetin powder C4 significantly reduces the morbidity and mortality of PRRSV-infected piglets. Compared with the blank control group, as the dose increases, the morbidity in PRRSV-infected piglets decreases to 20% to 60%, and the mortality among diseased piglets decreases to 10% to 40%. The experimental results fully demonstrate that the addition of the hesperetin preparation significantly increases the protection rate of piglets against PRRSV infection.

The expression levels of the PRRSV structural gene ORF7 in the brain and lungs of the piglets were detected. The detection results are shown in Table 7 below, where a, b, c, d indicate significant difference markers.

TABLE 7

| Sampled | Detected | Blank | 2 mg/kg | 5 mg/kg | 10 mg/kg |
|---|---|---|---|---|---|
| tissue | gene | group | group | group | group |
| Brain | ORF7 | 1.000 ± 0.122^{a} | 0.804 ± 0.113^{b} | 0.409 ± 0.091^{c} | 0.227 ± 0.034^{d} |
| Lung | ORF7 | 1.000 ± 0.143^{a} | 0.836 ± 0.148^{b} | 0.442 ± 0.083^{c} | 0.174 ± 0.027^{d} |

The results in Table 7 show that adding different doses of hesperetin powder C1 significantly reduces the expression level of the PRRSV ORF7 gene. The results fully indicate that the addition of the hesperetin preparation significantly reduces the replication of PRRSV in the brain and lungs.

In summary, the hesperetin preparation provided by the examples of the present disclosure possesses efficacy against porcine enveloped viruses such as PEDV, PRRSV, and ASFV. It can be used in the preparation of drugs and feed additives for treating and preventing porcine diseases caused by infections with porcine enveloped viruses such as PEDV, PRRSV, and ASFV. The dosage forms of the drugs and feed additives include oral liquids, granules, powders, tablets, and other formulations.

The present disclosure is described in detail above. Specific cases are used herein to illustrate the principle and implementation of the present disclosure, and the description of the above embodiments is only intended to help understand the core idea of the present disclosure. It should be noted that several improvements and alterations may also be made by those of ordinary skill in the art without departing from the principles of the present disclosure, which also fall within the scope of protection of the present disclosure.

1: A method for treating or preventing porcine enveloped virus infections, comprising administering a drug or a feed additive to a subject in need thereof, the drug or the feed additive comprises hesperetin, wherein the porcine enveloped virus comprises one or more of porcine epidemic diarrhea virus (PEDV), porcine reproductive and respiratory syndrome virus (PRRSV), and African swine fever virus (ASFV).

2-3. (canceled)

4: The method according to claim 1, wherein the drug or the feed additive is a composition comprising the hesperetin and an excipient, and the excipient comprises one or more of absolute ethanol, water, polyoxyethylene (20) sorbitan monooleate, lactose, defatted rice bran, corn cob powder, soluble starch, wheat bran, silicon dioxide, montmorillonite, vermiculite, maifan stone, and zeolite powder.

5: The method according to claim 4, wherein the composition comprising the hesperetin and the excipient is a solution formed by mixing the hesperetin with the absolute ethanol, the polyoxyethylene (20) sorbitan monooleate, and the water as three solvents, wherein the hesperetin has a concentration of 20 mg/mL to 100 mg/mL.

6: The method according to claim 4, wherein the composition comprising the hesperetin and the excipient is a solid mixture formed by mixing the hesperetin with the lactose and the soluble starch, wherein the hesperetin has a mass fraction of 45% to 55% in the solid mixture.

7: The method according to claim 4, wherein the composition comprising the hesperetin and the excipient is a powdered mixture formed by mixing the hesperetin with the soluble starch or the wheat bran, wherein the hesperetin has a mass fraction of 45% to 55% in the powdered mixture.

8: The method according to claim 4, wherein when the drug or the feed additive is the composition comprising the hesperetin and the excipient, a dosage form of the composition comprises one or more of an oral liquid, a granule, a tablet, and a powder.

9: The method according to claim 1, wherein a method for administering the drug or the feed additive comprises one or more of following (1) to (3):
(1) adding the hesperetin or a composition comprising the hesperetin and the excipient to a feed or drinking water, wherein a dosage of the hesperetin is 2 mg to 10 mg per kg of pig body weight;
(2) orally administering the hesperetin or the composition comprising the hesperetin and the excipient, wherein a dosage of the hesperetin is 2 mg to 10 mg per kg of pig body weight;
(3) adding the hesperetin or the composition comprising the hesperetin and the excipient to the feed for use at a dosage in which the content of the hesperetin is 0.01% to 0.03% of a total weight of the feed.
